(19) United States
(12) Patent Application Publication
(10) Pub. No.: US 2023/0181205 A1
(43) Pub. Date: Jun. 15, 2023

(54) TREATMENT TOOL

(71) Applicant: Olympus Corporation, Tokyo (JP)

(72) Inventors: Akira MIYAZAKI, Yokohama-shi (JP); Kazuhiro TANAKA, Tokyo (JP); Ojiro KITAMURA, Tokyo (JP)

(21) Appl. No.: 18/168,185

(22) Filed: Feb. 13, 2023

Related U.S. Application Data
(63) Continuation of application No. PCT/JP2020/031863, filed on Aug. 24, 2020.

(51) Int. Cl. A61B 17/28; A61B 17/285; A61B 17/22

(52) U.S. Cl. CPC A61B 17/282; A61B 17/285; A61B 17/2202; A61B 2017/2929

(57) ABSTRACT

A treatment tool includes: an insertion tube; a pair of jaws that are provided at a distal end of the insertion tube and that are flexible with respect to the insertion tube, the pair of jaws being configured to grasp living tissue by opening and closing each other; a pin that is provided on a rotation axis on which the jaws are caused to rotate with respect to the insertion tube; a shaft that has an outer circumferential surface and in which a hole into which the pin is inserted; a rod configured to be inserted into the insertion tube and move forward and backward along a longitudinal axis of the insertion tube; and a link mechanism that is linked to a distal end of the rod and that includes a plurality of arms linked with each other movably within a specific plane.

### CROSS-REFERENCE TO RELATED APPLICATION

This application is a continuation of International Application No. PCT/JP2020/031863, filed on Aug. 24, 2020, the entire contents of which are incorporated herein by reference.

### BACKGROUND

### 1. Technical Field

The present disclosure relates to a treatment tool.

### 2. Related Art

A treatment tool in which a pair of jaws that grasp living tissue, or the like, are flexible (oscillating move) has been known (refer to, for example, Japanese Laid-open Patent Publication No. 2018-020171).

The treatment tool described in Japanese Laid-open Patent Publication No. 2018-020171 includes a shaft and a link mechanism described below.

When viewed from a direction along a rotation axis on which the jaws are caused to flex, the shaft has an outer circumferential surface that is positioned on a circumference of a specific circle on the rotation axis.

The link mechanism is provided on a distal end side with respect to the shaft and is used to open or close the jaws.

### SUMMARY

In some embodiments, a treatment tool includes: an insertion tube that is tubular, the insertion tube being configured to be at least partly inserted into a body; a pair of jaws that are provided at a distal end of the insertion tube and that are flexible with respect to the insertion tube, the pair of jaws being configured to grasp living tissue by opening and closing each other; a pin that is provided on a rotation axis on which the jaws are caused to rotate with respect to the insertion tube; a shaft that has an outer circumferential surface and in which a hole into which the pin is inserted, the outer circumferential surface being positioned on a circumference of a specific circle on the rotation axis when viewed from a direction along the rotation axis on which the jaws are caused to flex with respect to the insertion tube; a rod configured to be inserted into the insertion tube and move forward and backward along a longitudinal axis of the insertion tube; and a link mechanism that is linked to a distal end of the rod and that includes a plurality of arms linked with each other movably within a specific plane, the link mechanism and the shaft being superimposed along the rotation axis with the specific surface being intersecting with the rotation axis.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of a treatment tool according to a first embodiment;

FIG. 2 is a diagram illustrating the configuration of the treatment tool according to the first embodiment;

FIG. 3 is a diagram illustrating the configuration of the treatment tool according to the first embodiment;

FIG. 4 is a diagram illustrating the configuration of the treatment tool according to the first embodiment;

FIG. 5 is a diagram illustrating the configuration of the treatment tool according to the first embodiment;

FIG. 6 is a diagram illustrating the configuration of the treatment tool according to the first embodiment;

FIG. 7 is a diagram illustrating a structure of connection between an end effector, a sheath, a flex mechanism, and an open-close mechanism;

FIG. 8 is a diagram illustrating the structure of connection of the end effector, the sheath, the flex mechanism, and the open-close mechanism;

FIG. 9 is a diagram illustrating the structure of connection of the end effector, the sheath, the flex mechanism, and the open-close mechanism;

FIG. 10 is a diagram illustrating the structure of connection of the end effector, the sheath, the flex mechanism, and the open-close mechanism;

FIG. 11 is a diagram illustrating the structure of connection of the end effector, the sheath, the flex mechanism, and the open-close mechanism;

FIG. 12 is a diagram illustrating a structure of joining of first and second wires and first and second flex rods;

FIG. 13 is a diagram illustrating a modification of the structure of joining of the first and second wires and the first and second flex rods;

FIG. 14 is a diagram illustrating a modification of the structure of joining of the first and second wires and the first and second flex rods;

FIG. 15 is a diagram for describing a change in a drive force in an open-close rod and a link mechanism;

FIG. 16 is a diagram for describing the change in the drive force in the open-close rod and the link mechanism;

FIG. 17 is a diagram for describing the change in the drive force in the open-close rod and the link mechanism;

FIG. 18 is a diagram for describing the change in the drive force in the open-close rod and the link mechanism;

FIG. 19 is a diagram for describing the change in the drive force in the open-close rod and the link mechanism;

FIG. 20 is a diagram illustrating a distal end part of a treatment tool according to a second embodiment;

FIG. 21 is a diagram illustrating the distal end part of the treatment tool according to the second embodiment;

FIG. 22 is a diagram illustrating the distal end part of the treatment tool according to the second embodiment;

FIG. 23 is a diagram illustrating an axis body according to a third embodiment;

FIG. 24 is a diagram illustrating an axis body according to a fourth embodiment; and

FIG. 25 is a diagram illustrating a configuration of a medical device according to a fifth embodiment.

### DETAILED DESCRIPTION

With reference to the accompanying drawings, modes for carrying out the disclosure (“embodiments” below) will be described below. Note that the embodiments do not limit the disclosure. Furthermore, in the illustration of the drawings, the same parts are denoted with the same reference numerals.

### First Embodiment

### Schematic Configuration of Treatment Tool

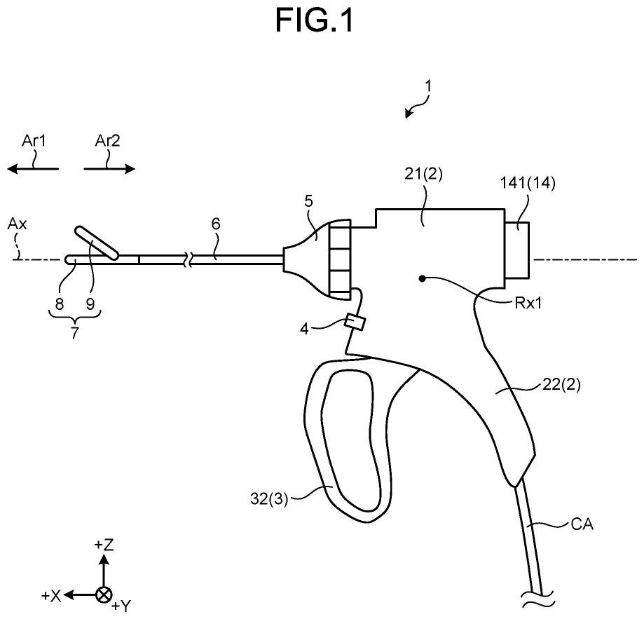

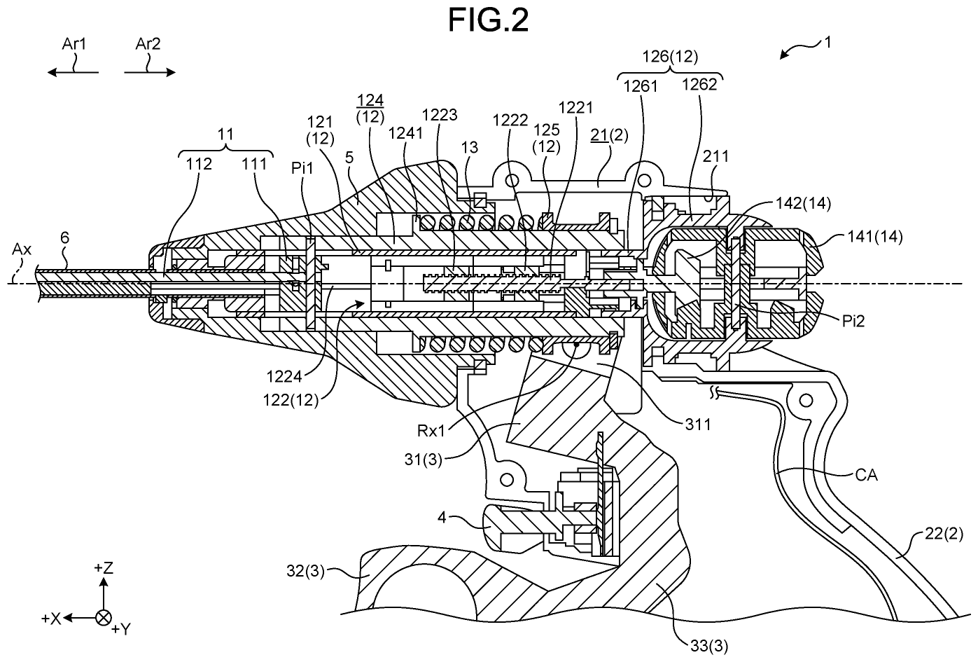

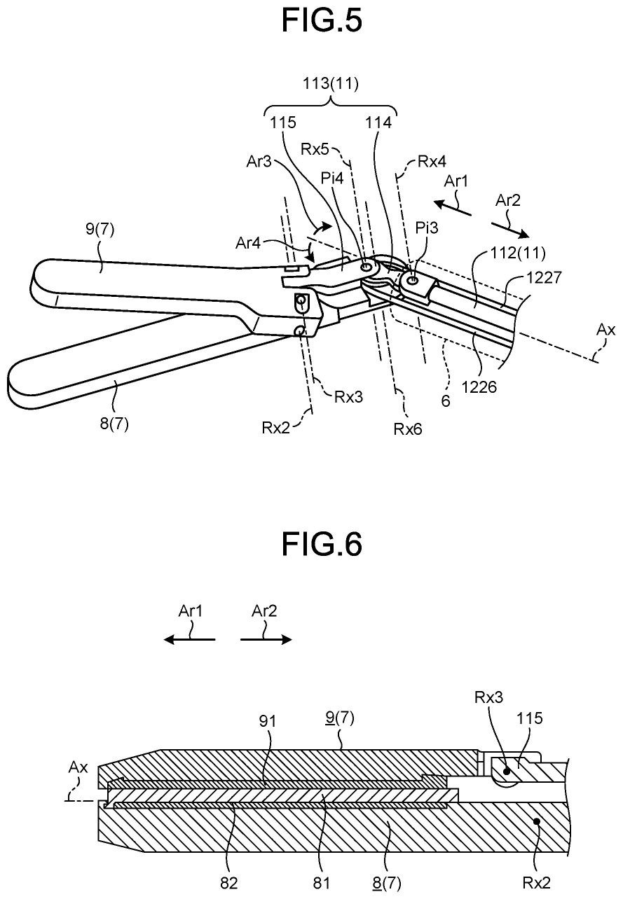

FIGS. 1 to 6 are diagrams illustrating a configuration of a treatment tool 1 according to a first embodiment. Specifically, FIG. 1 is a diagram illustrating an entire configuration of the treatment tool 1. FIGS. 2 to 4 are diagrams illustrating a configuration of a housing 2. FIG. 5 is a diagram illustrating a distal end part of the treatment tool 1. FIG. 6 is a cross-sectional view of the distal end part of the treatment tool 1, taken along a plane containing a center axis Ax (FIG. 1) of a sheath 6.

Note that, in FIG. 1 and FIG. 2, XYZ coordinate axes of an X-axis, a Y-axis, and a Z-axis that are orthogonal with one another are used. The X-axis is an axis parallel to a center axis Ax. The Y-axis is an axis orthogonal to the plane of drawing of FIG. 1 and FIG. 2. The Z-axis is an axis along a top-bottom direction in FIG. 1 and FIG. 2. One side (+X-axis side) along the center axis Ax is referred to as a distal end side Ar1 and the other side (−X-axis side) is referred to as a proximal end side Ar2.

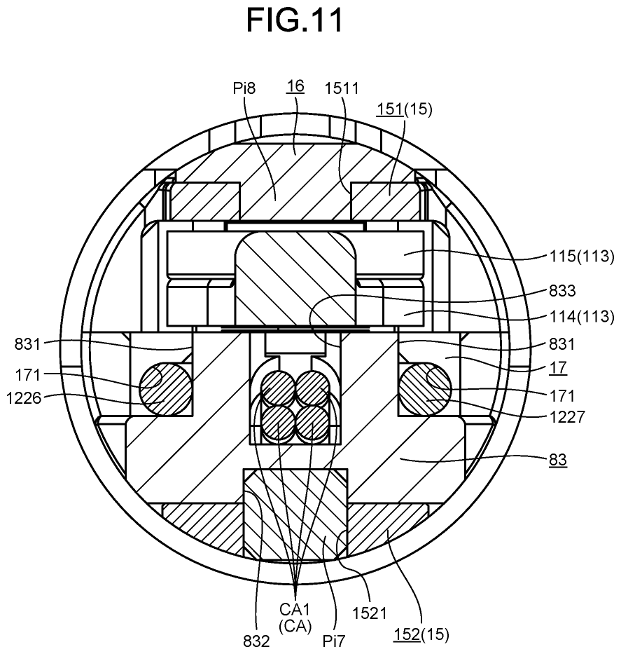

The treatment tool 1 treats a region to be treated (“subject region” below) in living tissue by applying a treatment energy to the subject region. Coagulating and cutting the subject region can be exemplified as the treatment. As illustrated in FIGS. 1 to 6, the treatment tool 1 includes the housing 2 (FIG. 1 and FIG. 2), a movable handle 3 (FIG. 1 and FIG. 2), a switch 4 (FIG. 1 and FIG. 2), a rotation knob 5 (FIGS. 1 to 4), the sheath 6, an end effector 7 (FIG. 1, FIG. 5 and FIG. 6), an electronic cable CA (four conductive cables CA1 (refer to FIG. 11)), a rotation member 12 (FIGS. 2 to 4), a coil spring 13 (FIGS. 2 to 4), and a flex operation unit 14 (FIGS. 1 to 4).

The housing 2 supports the entire treatment tool 1. As illustrated in FIG. 1 or FIG. 2, the housing 2 includes a housing body 21 that has an approximately cylindrical shape and that is coaxial with the center axis Ax and a fixed handle 22 that extends from the housing body 21 to the −Z-axis side (the bottom side in FIG. 1 and FIG. 2) and that is gripped by a practitioner.

As illustrated in FIG. 1 or FIG. 2, the movable handle 3 includes a handle base 31 (FIG. 2), a handle body 32, and a handle joint 33 (FIG. 2).

The handle base 31 is positioned in the housing 2 as illustrated in FIG. 2. A part of the handle base 31 on the +Z-axis side (upper side in FIG. 1 and FIG. 2) is supported axially on the housing 2 such that the part is rotatable about a first rotation axis Rx1 (FIG. 1 and FIG. 2). In an end portion of the handle base 31 on the +Z-axis side, a pair of engaging parts 311 (FIG. 2) that are opposed to each other along the Y-axis direction are provided with a slider 125 (FIG. 2) interposed in between, which is a slider that forks and protrudes to the +Z-axis side and forms the rotation member 12. The engaging parts 311 are parts that are engaged with the slider 125. Note that FIG. 2 illustrates only the engaging part 311 in the +Y-axis direction (depth direction with respect to the plane of drawing of FIG. 2) in the engaging parts 311.

The handle body 32 is a part that receives each of a closing operation and an opening operation performed by the practitioner and, as illustrated in FIG. 1 or FIG. 2, the handle body 32 is positioned outside the housing 2.

As illustrated in FIG. 2, the handle joint 33 is a part that is arranged straddling the inside and outside the housing 2 and that connects the handle base 31 and the handle body 32.

As illustrated in FIG. 1 or FIG. 2, the switch 4 is arranged in a state of being exposed to the outside from a side surface of the fixed handle 22 on the distal end side Ar1 and receives an output start operation performed by the practitioner. The output start operation is an operation of pressing down the switch 4 and is an operation of starting application of a treatment energy to the subject region. The switch 4 outputs an operation signal corresponding to the output start operation to an external control device (not illustrated in the drawings) via the electronic cable CA (FIG. 1 and FIG. 2).

The rotation knob 5 has an approximately cylindrical shape extending along the center axis Ax and is supported by the housing body 21 rotatably on the center axis Ax in a posture such that the rotation knob 5 is coaxial with the center axis Ax. The rotation knob 5 receives a rotation operation performed by the practitioner. The rotation operation causes the rotation knob 5 to rotate on the center axis Ax with respect the housing body 21.

The sheath 6 corresponds to an insertion tube. An end portion of the sheath 6 on the proximal end side Ar2 is inserted into the rotation knob 5 and is fixed to an inner surface of the rotation knob 5 by welding, or the like. In other words, the sheath 6 rotates on the center axis Ax together with the rotation knob 5 according to the rotation operation performed by the practitioner on the rotation knob 5.

As illustrated in FIG. 1, FIG. 5 or FIG. 6, the end effector 7 includes first and second graspers 8 and 9. The first and second graspers 8 and 9 correspond to a pair of jaws.

The first grasper 8 has an elongated shape that extends along the center axis Ax.

As illustrated in FIG. 6, the first grasper 8 includes a first electrode 81 and a heater 82 in its end portion that is on the distal end side Ar1 and that is opposed to the second grasper 9.

The first electrode 81 is made of a conductive material that has high thermal conductivity, such as copper, and has an elongated shape that extends along the center axis Ax. The first electrode 81 is provided in a state of being exposed to the outside in the first grasper 8.

In the first grasper 8, the heater 82 is provided in a state of being buried inside because of the first electrode 81. Supply of an electric power causes the heater 82 to generate heat and heat the first electrode 81. For example, a sheet heater obtained by patterning a conductive pattern on a substrate made of polyimide, or the like, a ceramic heater obtained by patterning a conductive pattern on a ceramic substrate, such as aluminum nitride, or another printed heater can be exemplified as the heater 82.

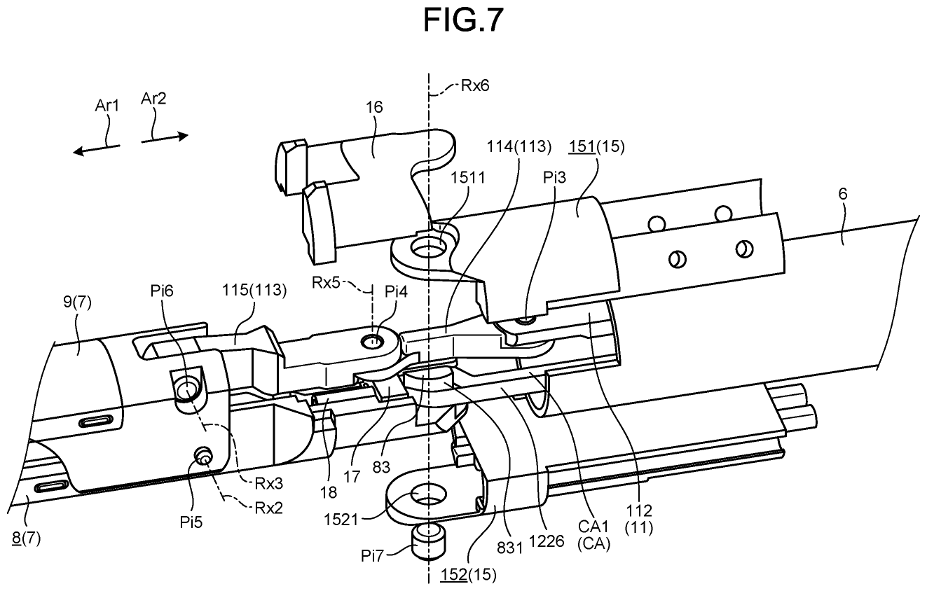

In an end portion of the first grasper 8 on the proximal end side Ar2, a shaft 83 (refer to FIG. 7) for causing the end effector 7 to take a flex move (rotation move) on a sixth rotation axis Rx6 (FIG. 5) with respect to the sheath 6 is provided. The sixth rotation axis Rx6 corresponds to a rotation axis and is an axis orthogonal to the center axis Ax. The sixth rotation axis Rx6 is an axis along a direction in which the second grasper 9 opens and closes with respect to the first grasper 8 (in the top-bottom direction in FIG. 6).

Note that a detailed configuration of the shaft 83 will be described in “Structure of Connection of End Effector, Sheath, Flex mechanism and Open-close Mechanism” to be described below.

The second grasper 9 has an elongated shape that extends along the center axis Ax. A length dimension of the second grasper 9 in a longitudinal direction is shorter than a length dimension of the first grasper 8 in the longitudinal direction. A fifth pin Pi5 (refer to FIG. 7) that is cylindrical and that bridges the first and second graspers 8 and 9 allows an end of the second grasper 9 on the proximal end side Ar2 to be pivotally supported on the first grasper 8 rotatably on the second rotation axis Rx2 (FIG. 5 and FIG. 6). The second grasper 9 rotates on the second rotation axis Rx2 and accordingly the second grasper 9 opens and closes with respect to the first grasper 8, which makes it possible to grasp the subject region between the first and second graspers 8 and 9.

In a portion of the second grasper 9 that is opposed to the first electrode 81, a second electrode 91 (FIG. 6) made of a conductive material is provided in a state of being exposed to the outside. The first and second electrodes 81 and 91 correspond to electrodes.

In an end portion of the second grasper 9 on the proximal end side Ar2, a sixth pin Pi6 (refer to FIG. 7) to which an open-close mechanism 11 forming the rotation member 12 is connected is provided. The sixth pin Pi6 extends along a third rotation axis Rx3 that is parallel with the second rotation axis Rx2.

The four conductive cables CA1 are conductive cables forming part of the electronic cable CA that is drawn in the housing 2 from an end portion of the fixed handle 22 on the −Z-axis side. The four conductive cables CA1 are drawn from the end portion of the fixed handle 22 on the −Z-axis side into the sheath 6 through the housing 2 and the rotation knob 5. Two of the four conductive cables CA1 are electrically connected to the first and second electrodes 81 and 91. The remaining two conductive cables CA1 are electrically connected to the heater 82.

The external control device (not illustrated in the drawings) runs as follows according to the output start operation performed by the practitioner on the switch 4.

The control device supplies a high-frequency power between the first and second electrodes 81 and 91 via the two conductive cables CA1. Accordingly, a high-frequency current flows into the subject region that is graspted between the first and second electrodes 81 and 91. In other words, a high-frequency energy is applied as a treatment energy to the subject region. The subject region is thus treated.

The control device supplies a power to the heater 82 via the two conductive cables CA1. Accordingly, heat from the heater 82 is transmitted to the subject region that is grasped between the first and second electrodes 81 and 91 via the first electrode 81. In other words, a thermal energy is applied as a treatment energy to the subject region. The subject region is thus treated.

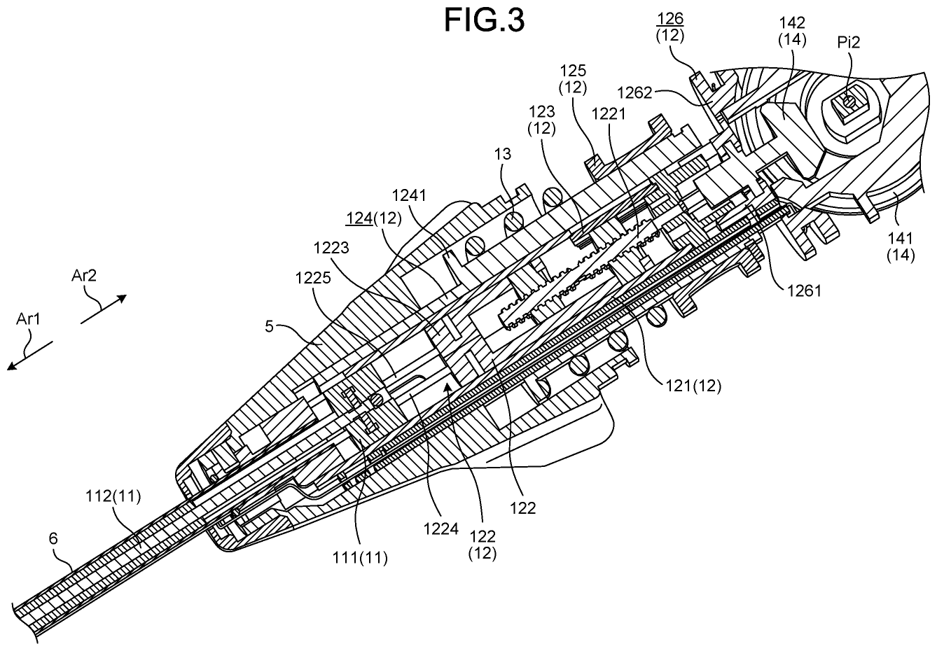

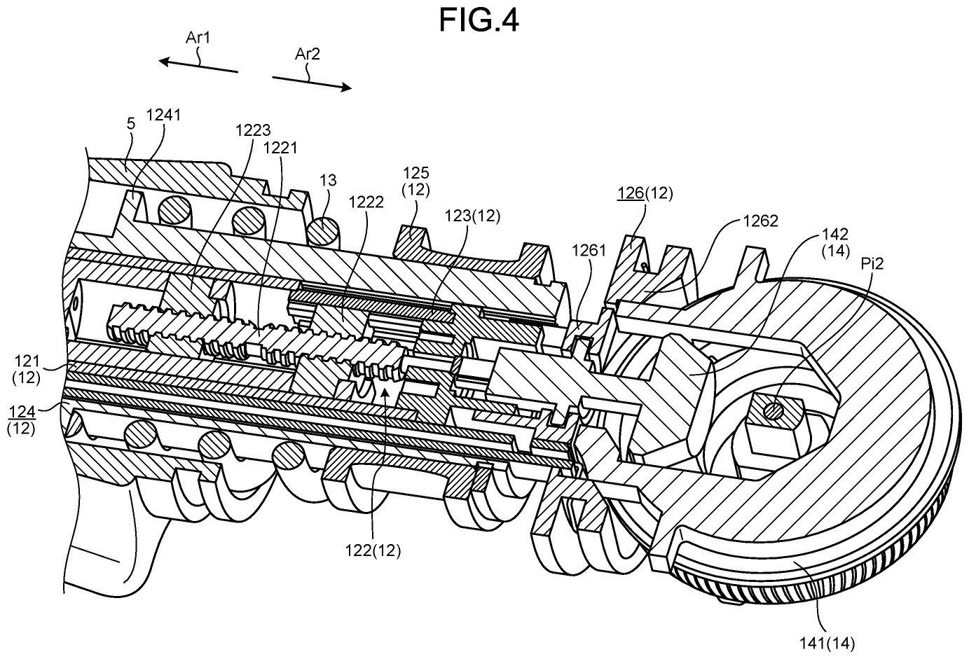

According to the rotation operation performed by the practitioner on the rotation knob 5, the rotation member 12 rotates on the center axis Ax together with the rotation knob 5. As illustrated in FIGS. 2 to 4, the rotation member 12 includes a first support member 121, a flex mechanism 122, a rotation regulation member 123 (FIG. 3 and FIG. 4), a slider receiver 124, the slider 125, an open-close mechanism 11 (FIG. 2 and FIG. 3), and a second support member 126.

As illustrated in FIGS. 2 to 4, the first support member 121 has a cylindrical shape extending along the center axis Ax and is arranged in a posture such that the first support member 121 is coaxial with the center axis Ax. More specifically, the first support member 121 is inserted into the rotation knob 5 and the housing body 21, straddling the rotation knob 5 and the housing body 21. An end of the first support member 121 on the distal end side Ar1 is fixed to the inner surface of the rotation knob 5 by welding, or the like.

The first support member 121 described above supports part of the flex mechanism 122 and part of the open-close mechanism 11 in the first support member 121.

Note that the configurations of the flex mechanism 122 and the rotation regulation member 123 will be described together with the configuration of the flex operation unit 14.

As illustrated in FIGS. 2 to 4, the slider receiver 124 has a cylindrical shape extending along the center axis Ax and is arranged in a posture such that the slider receiver 124 is coaxial with the center axis Ax. More specifically, in a state where the slider receiver 124 is inserted into the coil spring 13 and the first support member 121 is inserted into the slider receiver 124, the slider receiver 124 is arranged movably with respect to the first support member 121 along the center axis Ax. An end of the slider receiver 124 on the distal end side Ar1 is fixed to the open-close mechanism 11 that is held in the first support member 121 with a first pin Pi1 (FIG. 2) in a state where move of the end of the slider receiver 124 along the center axis Ax with respect to the first support member 121 is allowed and rotation on the center axis Ax is restricted.

As illustrated in FIGS. 2 to 4, in the slider receiver 124, a protruding portion 1241 that protrudes from an outer circumferential surface and that extends over the circumference in a circumferential direction around the center axis Ax is provided.

As illustrated in FIGS. 2 to 4, the slider 125 has an approximately cylindrical shape extending along the center axis Ax and is arranged in a posture such that the slider 125 is coaxial with the center axis Ax. More specifically, the slider 125 is arranged movably with respect to the slider receiver 124 along the center axis Ax with the slider receiver 124 being inserted into the slider 125. As described above, the slider 125 is engaged with the movable handle 3 by the engaging parts 311.

The coil spring 13 has a function of applying a drive force to the second grasper 9 from the first and second graspers 8 and 9 forming the end effector 7 according to the close operation and the open operation performed by the practitioner on the movable handle 3. The drive force is a drive force for opening and closing the second grasper 9 with respect to the first grasper 8. As illustrated in FIGS. 2 to 4, the coil spring 13 is arranged in a state of being interposed between the protruding portion 1241 and the slider 125, with the slider receiver 124 being inserted into the coil spring 13.

The open-close mechanism 11 is a mechanism that opens and closes the second grasper 9 with respect to the first grasper 8. As illustrated in FIG. 2, FIG. 3 or FIG. 5, the open-close mechanism 11 includes an open-close joint 111 (FIG. 2 and FIG. 3), an open-close rod 112 (FIG. 2 and FIG. 3), and a link mechanism 113 (FIG. 5).

As illustrated in FIG. 2, the open-close joint 111 is a part that is fixed to the slider receiver 124 with the first pin Pi1 and is held in the first support member 121 movably along the center axis Ax.

The open-close rod 112 corresponds to a rod. The open-close rod 112 is an elongated member that extends along the center axis Ax and is inserted into the sheath 6. As illustrated in FIG. 2 or FIG. 3, an end of the open-close rod 112 on the proximal end side Ar2 protrudes to the outside of the sheath 6 and is inserted into the first support member 121 and is fixed to the open-close joint 111. In other words, the open-close rod 112 is movable along the center axis Ax together with the open-close joint 111.

The link mechanism 113 is a mechanism that links the open-close rod 112 and the second grasper 9 (the sixth pin Pi6).

Note that a detailed configuration of the link mechanism 113 will be described in “Structure of Connection of End Effector, Sheath, Flex mechanism and Open-close Mechanism” to be described below.

The slider 125, the slider receiver 124, and the open-close mechanism 11 move as described below according to an operation performed by the practitioner on the movable handle 3.

According to the close operation performed by the practitioner on the movable handle 3, the slider 125 is pushed into by the engaging parts 311 along the center axis Ax toward the distal end side Ar1. The slider receiver 124 receives a push force (drive force for opening and closing the second grasper 9 with respect to the first grasper 8) from the slider 125 toward the distal end side Ar1 via the coil spring 13. Furthermore, the open-close mechanism 11 moves in association with the slider receiver 124 toward the distal end side Ar1. The open-close mechanism 11 transmits the drive force to the second grasper 9. Accordingly, the second grasper 9 rotates on the second rotation axis Rx2 in a direction in which the second grasper 9 gets close to the first grasper 8 (close direction).

On the other hand, when the open operation on the movable handle 3 is performed by the practitioner, the slider 125, the slider receiver 124, and the open-close mechanism 11 move in a direction inverse to the above-described direction. Accordingly, the second grasper 9 rotates on the second rotation axis Rx2 in a direction in which the second grasper 9 separates from the first grasper 8 (open direction).

The second support member 126 is a member that supports the flex operation unit 14. As illustrated in FIGS. 2 to 4, the second support member 126 includes a fitting part 1261 and a support member body 1262.

As illustrated in FIGS. 2 to 4, the fitting part 1261 is formed into a cylindrical shape having an outer diameter dimension approximately equal to an inner diameter dimension of the slider receiver 124 and is fitted into the slider receiver 124, thereby linking to the slider receiver 124.

As illustrated in FIGS. 2 to 4, the support member body 1262 is formed in an approximately cylindrical shape having an outer diameter dimension larger than an outer diameter dimension of the slider receiver 124 and is formed integrally with the fitting part 1261 and in a posture such that the support member body 1262 is coaxial with the fitting part 1261. The support member body 1262 internally supports the flex operation unit 14 and is exposed to the outside of the housing body 21 from a proximal end opening 211 (FIG. 2) of the housing body 21 on the proximal end side Ar2.

As illustrated in FIGS. 1 to 4, the flex operation unit 14 includes a flex operation unit body 141 and a rotation converter 142 (FIGS. 2 to 4).

As illustrated in FIGS. 1 to 4, the flex operation unit body 141 has an overall approximately cylindrical shape. A cylindrical second pin Pi2 (FIGS. 2 to 4) is inserted on the center axis of the flex operation unit body 141. Furthermore, with the second pin Pi2, the flex operation unit body 141 is held in the support member body 1262 rotatably on the second pin Pi2. In that state, the flex operation unit body 141 is positioned on the center axis Ax. The flex operation unit body 141 receives a flex operation (an operation of causing the end effector 7 to take a flex move with respect to the sheath 6) performed by the practitioner. According to the flex operation, the flex operation unit body 141 rotates on the second pin Pi2 with respect to the support member body 1262.

As illustrated in FIGS. 2 to 4, the rotation converter 142 links to each of the flex operation unit body 141 and the flex mechanism 122. The rotation converter 142 converts the rotation on the second pin Pi2 according to the flex operation performed by the practitioner on the flex operation unit body 141 into rotation on the center axis Ax. In other words, the rotation converter 142 rotates on the center axis Ax according to the flex operation. A bevel gear, or the like, can be exemplified as the rotation converter 142.

The flex mechanism 122 is a mechanism that causes the end effector 7 to take a flex move with respect to the sheath 6 and, as illustrated in FIGS. 2 to 5, the flex mechanism 122 includes a rotation shaft 1221 (FIGS. 2 to 4), first and second drivers 1222 and 1223 (FIGS. 2 to 4), first and second flex rods 1224 and 1225 (FIG. 2 and FIG. 3), and first and second wires 1226 and 1227 (FIG. 5).

The rotation shaft 1221 is a cylindrical elongated member that extends along the center axis Ax and is inserted into the first support member 121 in a posture such that the rotation shaft 1221 is coaxial with the center axis Ax. An end of the rotation shaft 1221 on the proximal end side Ar2 is fixed to the rotation converter 142. In other words, the rotation shaft 1221 rotates on the center axis Ax together with the rotation converter 142 according to the flex operation performed by the practitioner on the flex operation unit body 141.

Each of the first and second drivers 1222 and 1223 is engaged with the rotation shaft 1221 because of an engagement structure in which the first and second drivers 1222 and 1223 that are threaded oppositely. The first and second drivers 1222 and 1223 move in association with rotation of the rotation shaft 1221 on the center axis Ax and are held in the first support member 121 movably in inverse directions along the center axis Ax.

The first and second flex rods 1224 and 1225 are elongated members each of which extends along the center axis Ax and is inserted into the sheath 6. An end of the first flex rod 1224 on the proximal end side Ar2 protrudes to the outside of the sheath 6, is inserted into the first support member 121, and is fixed to the first driver 1222. On the other hand, an end of the second flex rod 1225 on the proximal end side Ar2 protrudes to the outside of the sheath 6, is inserted into the first support member 121, and is fixed to the second driver 1223. In other words, the first and second rods 1224 and 1225 are movable along the center axis Ax together with the first and second drivers 1222 and 1223.

The first and second wires 1226 and 1227 are wires made of a resin material or a metal material. An end portion of the first wire 1226 on the proximal end side Ar2 is joined to an end portion of the first flex rod 1224 on the distal end side Ar1 in the sheath 6. An end portion of the first wire 1226 on the distal end side Ar1 protrudes to the outside of the sheath 6 and is connected to the first grasper 8. On the other hand, an end portion of the second wire 1227 on the proximal end side Ar2 is joined to an end portion of the second flex rod 1225 on the distal end side Ar1 in the sheath 6. An end portion of the second wire 1227 on the distal end side Ar1 protrudes to the outside of the sheath 6 and is connected to the first grasper 8.

Note that details of the structure of connection of the first and second wires 1226 and 1227 and the first grasper 8 will be described in “Structure of Connection of End effector, Sheath, Flex Mechanism and Open-Close Mechanism” described below. Details of the structure of joining of the first and second wires 1226 and 1227 and the first and second flex rods 1224 and 1225 will be described in “Structure of Joining of First and Second Wires and First and Second Flex Rods” described below.

The flex mechanism 122 moves as described below according to the flex operation performed by the practitioner on the flex operation unit body 141.

First of all, the case where the practitioner causes the flex operation unit body 141 to rotate on the second pin Pi2 in a first direction (flex operation) is assumed. In this case, the operation force is transmitted from the flex operation unit body 141 to the first and second rods 1224 and 1225 via the rotation converter 142, the rotation shaft 1221, and the first and second drivers 1222 and 1223. The second flex rod 1225 moves along the center axis Ax toward the proximal end side Ar2 and pulls the second wire 1227 toward the proximal end side Ar2. On the other hand, the first flex rod 1224 moves along the center axis Ax to the proximal end side Ar1 and, according to the pulling of the second wire 1227 toward the proximal end side Ar2, sends the first wire 1226 toward the distal end side Ar1. Accordingly, the end effector 7 rotates on the sixth rotation axis Rx6 with respect to the sheath 6 in a first flex direction Ar3 (FIG. 5). In other words, the end effector 7 takes the flex move.

The case where the practitioner causes the flex operation unit body 141 to rotate on the second pin Pi2 in a direction opposite to the first direction (flex operation) is assumed next. In this case, opposite to the above-described case, the first flex rod 1224 moves along the center axis Ax to the proximal end side Ar2 and pulls the first wire 1226 toward the proximal end side Ar2. On the other hand, opposite to the above-described case, the second flex rod 1225 moves along the center axis Ax toward the distal end side Ar1 and sends the second wire 1227 toward the distal end side Ar1. Accordingly, the end effector 7 rotates on the sixth rotation axis Rx6 with respect to the sheath 6 in a second flex direction Ar4 (FIG. 5) that is opposite to the first flex direction Ar3. In other words, the end effector 7 takes the flex move.

The rotation regulation member 123 is a member that regulates rotation of the first driver 1222 on the center axis Ax. As illustrated in FIG. 3 or FIG. 4, in the first support member 121, the rotation regulation member 123 is fixed to the first driver 1222 in a position opposed to a direction orthogonal to the center axis Ax. While move of the first driver 1222 along the center axis Ax with respect to the rotation regulation member 123 is allowed, rotation of the first driver 1222 on the center axis Ax is regulated. The second driver 1223 abuts on the first driver 1222 and accordingly rotation on the center axis Ax is regulated.

### Structure of Connection of End Effector, Sheath, Flex Mechanism, and Open-close Mechanism

A structure of connection of the end effector 7, the sheath 6, the flex mechanism 122, and the open-close mechanism 11 will be described next.

FIGS. 7 to 11 are diagrams illustrating the structure of connection of the end effector 7, the sheath 6, the flex mechanism 122, and the open-close mechanism 11. Specifically, FIGS. 7 to 10 are exploded perspective views illustrating the structure of connection. FIG. 11 is a cross-sectional view of the structure of connection taken along a plane containing the sixth rotation axis Rx6 and orthogonal to the center axis Ax.

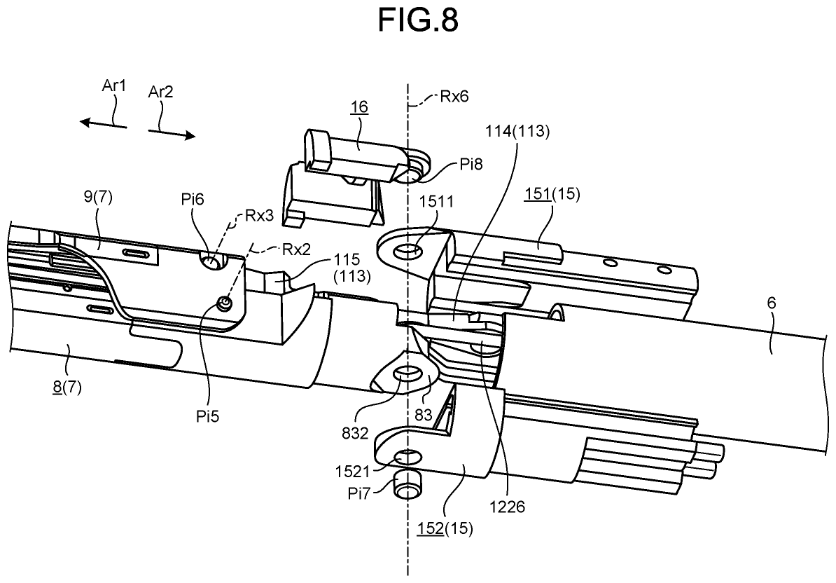

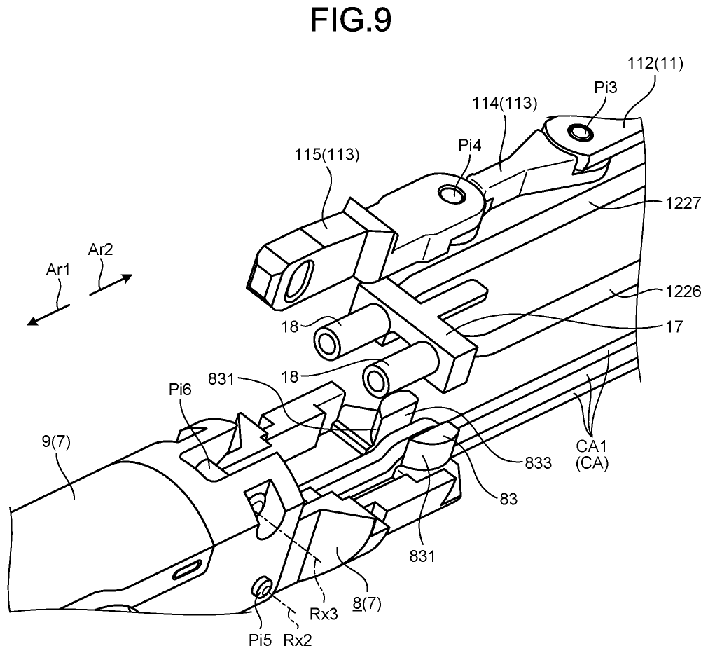

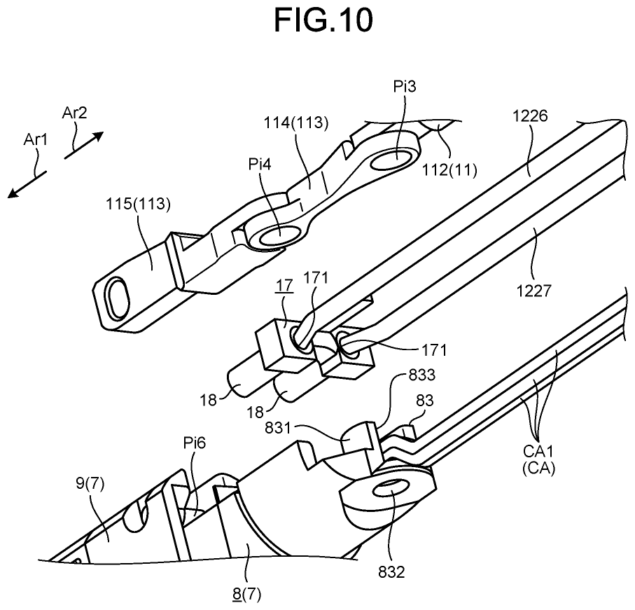

In the first embodiment, as illustrated in FIGS. 7 to 11, the shaft 83, the link mechanism 113, a sheath-side connector 15 (FIG. 7, FIG. 8 and FIG. 11), an end-effector-side connector 16 (FIG. 7, FIG. 8 and FIG. 11), a holder 17 (FIG. 7 and FIGS. 9 to FIG. 11), and a pair of fall stoppers 18 (FIG. 7, FIG. 9 and FIG. 10) are used as the structure of connection of the end effector 7, the sheath 6, the flex mechanism 122, and the open-close mechanism 11.

As illustrated in FIGS. 7 to 11, the shaft 83 is provided in the end portion of the first grasper 8 on the proximal end side Ar2.

According to FIGS. 7 to 9, when viewed from a direction along the sixth rotation axis RX6, a pair of outer circumferential surfaces 831 that are positioned on a circumference of a specific circle on the sixth rotation axis Rx6 are provided on an upper side in the shaft 83.

According to FIGS. 7 to 9, a shaft receiving hole 832 (FIG. 8) that extends toward the upper side and into which a seventh pin Pi7 that is cylindrical is inserted is provided on a lower side in the shaft 83.

Furthermore, in the shaft 83, as illustrated in FIG. 9, a path 833 that extends linearly along a longitudinal direction of the first grasper 8 is provided between the outer circumferential surfaces 831. In the first embodiment, the path 833 passes through the sixth rotation axis Rx6. The path 833 is formed by a groove. As illustrated in FIGS. 9 or FIG. 11, the four conductive cables CA1 are arranged in the path 833.

As illustrated in FIGS. 7 to 11, the link mechanism 113 includes a proximal end arm 114 and a distal end arm 115.

The proximal end arm 114 is an elongated member. An end portion of the proximal end arm 114 on the proximal end side Ar2 is linked to the open-close rod 112 with a third pin Pi3 that is cylindrical rotatably on a fourth rotation axis Rx4. The fourth rotation axis Rx4 is an axis parallel to the sixth rotation axis Rx6.

The distal end arm 115 is an elongated member. An end portion of the distal end arm 115 on the proximal end side Ar2 is linked to an end portion of the proximal end arm 114 on the distal end side Ar1 with a fourth pin Pi4 that is cylindrical rotatably on a fifth rotation axis Rx5. The fifth rotation axis 5 is an axis parallel to the fourth rotation axis Rx4. The distal end arm 115 is linked to the second grasper 9 with the sixth pin Pi6 rotatably on the third rotation axis Rx3.

In other words, in the link mechanism 113, the proximal end arm 114 and the distal end arm 115 are linked mutually movably on a specific plane orthogonal to the sixth rotation axis Rx6. While moving on the specific plane, the link mechanism 113 transmits a drive force transmitted via the open-close rod 112 to the second grasper 9 in accordance with the flex move of the end effector 7 with respect to the sheath 6.

As illustrated in FIG. 11, the link mechanism 113 described above is arranged on the shaft 83 along the sixth rotation axis RX6 in a superimposed manner. In other words, the link mechanism 113 and the shaft 83 are superimposed along the direction in which the second grasper 9 opens and closes with respect to the first grasper 8. When viewed from the direction along the sixth rotation axis Rx6, the shaft 83 is positioned between the proximal end arm 114 and the distal end arm 115.

As illustrated in FIG. 7, FIG. 8 or FIG. 11, the sheath-side connector 15 includes a first sheath-side connector 151 and a second sheath-side connector 152.

In a state of sandwiching the end portion of the sheath 6 on the distal end side Ar1 in the top-bottom direction in FIG. 7, the first sheath-side connector 151 and the second sheath-side connector 152 are fixed to the end portion.

In an end portion of the first sheath-side connector 151 on the distal end side Ar1, a shaft receiving hole 1511 into which an eighth pin Pi8 described below is inserted is provided. Similarly, in an end portion of the second sheath-side connector 152 on the distal end side Ar1, a shaft receiving hole 1521 into which the seventh pin Pi7 is inserted is provided.

As illustrated in FIG. 7, FIG. 8 or FIG. 11, in a state of covering the link mechanism 113 from the upper side in FIG. 7, the end-effector-side connector 16 is fixed to the end portion of the first grasper 8 on the proximal end side Ar2.

As illustrated in FIG. 8, the eighth pin Pi8 that is cylindrical and that is inserted into the shaft receiving hole 1511 is provided in the end-effector-side connector 16.

The seventh and eighth pins Pi7 and Pi8 enable the end effector 7 to take the flex move on the sixth rotation axis Rx6 with respect to the sheath 6.

As illustrated in FIGS. 7 to 11, in a state of sandwiching the shaft 83, the first and second wires 1226 and 1227 are arranged along the outer circumferential surfaces 831, respectively. In a state of being separate from each other in a distance smaller than the diameter of the above-described specific circle that is formed by the outer circumferential surfaces 831, each of the first and second wires 1226 and 1227 extends toward the distal end of the end effector 7. In this state, the four conductive cables CA1 are arranged between the first and second wires 1226 and 1227 in a state of being separate from each other.

In the first embodiment, even in the case where the end effector 7 flexes toward the first flex direction Ar3 or the second flex direction Ar4 at a flex angle up to approximately 90 degrees with respect to the center axis Ax, the distance smaller than the diameter of the specific circle described above is set at a distance that maintains the state in which the first and second wires 1226 and 1227 abut on the outer circumferential surfaces 831.

The holder 17 is used to fix the first and second wires 1226 and 1227 to the first grasper 8. As illustrated in FIG. 10 and FIG. 11, a pair of insertion holes 171 into which the first and second wires 1226 and 1227 are inserted, respectively, are provided in the holder 17. The holder 17 is fixed to a portion of the first grasper 8 on the distal end side Ar1 with respect to the shaft 83.

The fall stoppers 18 consist of pipes and are attached to the distal ends of the first and second wires 1226 and 1227, respectively. The fall stoppers 18 thus prevent the first and second wires 1226 and 1227 from falling off the insertion holes 171 in the holder 17.

### Structure of Joining of First and Second Wires and First and Second Flex Rods

The structure of joining of the first and second wires 1226 and 1227 and the first and second flex rods 1224 and 1225 will be described next.

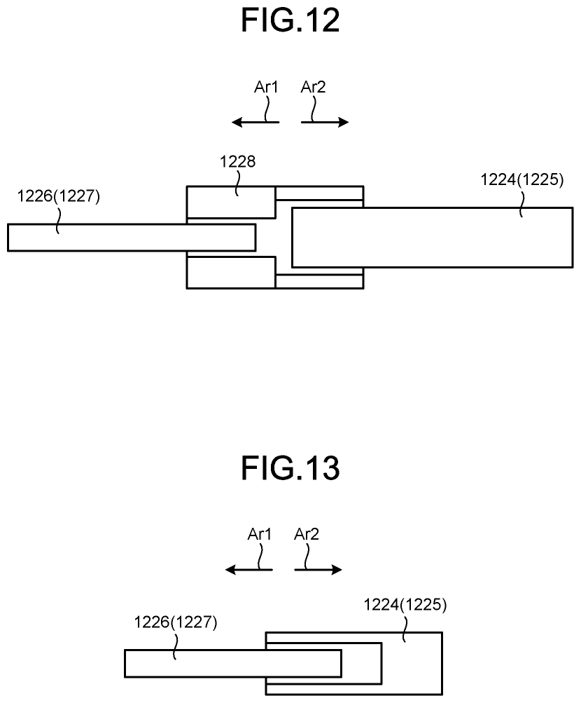

FIG. 12 is a diagram illustrating a structure of joining of the first and second wires 1226 and 1227 and the first and second flex rods 1224 and 1225.

As illustrated in FIG. 12, the first wire 1226 (the second wire 1226) is joined to the first flex rod 1224 (the second flex rod 1225) by swaging or welding using an intermediate pipe 1228.

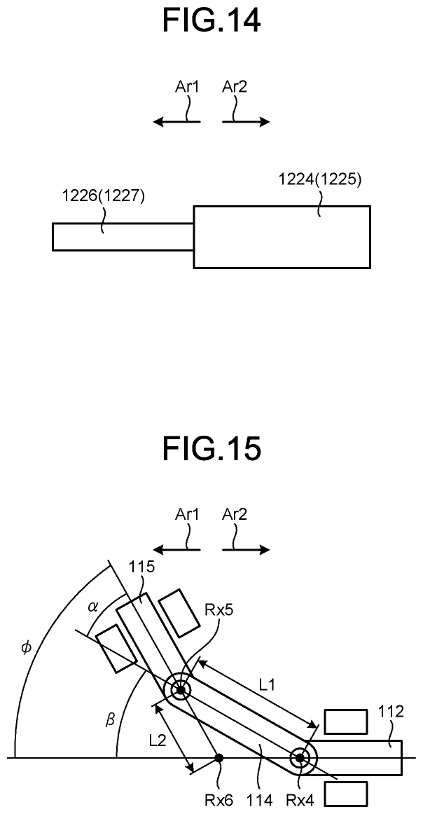

FIG. 13 and FIG. 14 are diagrams illustrating modifications of the structure of joining of the first and second wires 1226 and 1227 and the first and second flex rods 1224 and 1225.

Note that the structure of joining of the first and second wires 1226 and 1227 and the first and second flex rods 1224 and 1225 is not limited to the above-described structure of joining using the intermediate pipe 1228.

For example, as presented in the modifications in FIG. 13 and FIG. 14, the first wire 1226 (the second wire 1227) may be directly joined to the first flex rod 1224 (the second flex rod 1225) by swaging or welding.

### As for Change in Drive Force in Open-close Rod and Link Mechanism

A change in a drive force in the open-close rod 112 and the link mechanism 113 will be described next.

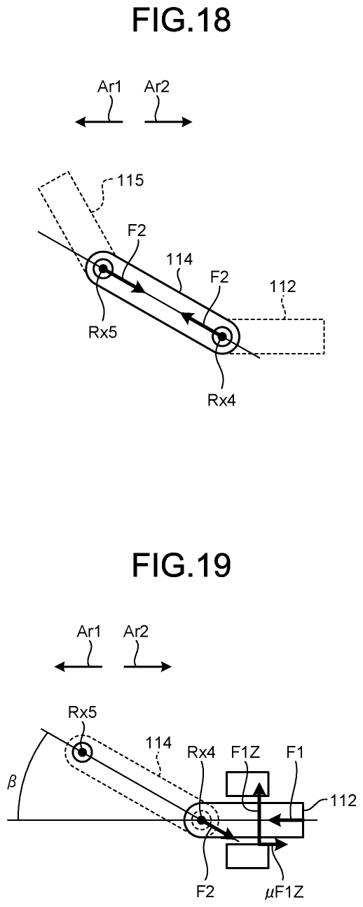

FIGS. 15 to 19 are diagrams for describing a change in the drive force in the open-close rod 112 and the link mechanism 113.

A flex angle φ of the end effector 7 with respect to the sheath 6 is expressed by Equation (1) below.

ϕ=α+β (1)

In Equation (1), α denotes an angle formed by the distal end arm 115 with respect to the proximal end arm 114 (FIG. 15). β denotes an angle formed by the proximal end arm 114 with respect to the open-close rod 112 (FIG. 15).

Equation (2) below is derived from FIG. 15.

L1·sin β=L2·sin ϕ (2)

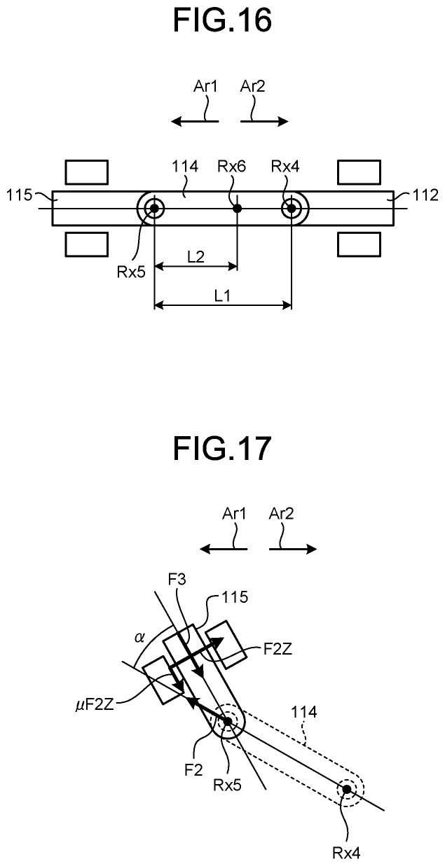

In Equation (2), L1 denotes a length dimension between the fourth and fifth rotation axes Rx4 and Rx5 (FIG. 15 and FIG. 16). L2 denotes a length dimension between the fifth and sixth rotation axes Rx5 and Rx6 (FIG. 15 and FIG. 16).

Modifying Equation (2) leads to Equation (3) below.

$$β = {sin}^{- 1} ( \frac{L 2}{L 1} \cdot sin ϕ ) \qquad ( 3 )$$

In other words, Equation (1) and Equation (3) present that α and β at the time of the flex angle φ is determined by L2/L1.

The relationship of forces applied to the open-close rod 112 and the link mechanism 113 is represented by Equations (4) to (7) below.

F3=F2·cos α−μF2Z (4)

F2Z=F2·sin α (5)

F1=F2·cos β+μF1Z (6)

F1Z=F2·sin β (7)

In Equation (4), F3 denotes a drive force that is transmitted from the distal end arm 115 to the second grasper 9 (FIG. 17). In Equations (4) to (7), F2 denotes a drive force that is applied between the open-close rod 112 and the distal end arm 115 (FIGS. 17 to 19). In Equation (4) and Equation (5), F2Z denotes a normal force applied to the distal end arm 115 (FIG. 17). In Equation (4) and Equation (6), μ denotes a friction coefficient. In Equation (6), F1 denotes a drive force that is transmitted to the open-close rod 112 (FIG. 19). In Equation (6) and Equation (7), F1Z denotes a normal force applied to the open-close rod 112.

Putting Equations (4) to (7) together leads to Equation (8) below.

$$\frac{F 3}{F 1} = \frac{( cos α - μ sin α )}{( cos β + μ sin β )} \qquad ( 8 )$$

In other words, Equation (8) presents that a rate of change in the drive force (F3/F1) depends on α and β. As described above, α and β depend on L2/L1 at the time of the flex angle φ, which presents that the rate of change in the drive force (F3/F1) at the time of the flex angle φ depends on L2/L1.

The case of a structure in which the end effector 7 is flexible at a flex angle φ up to 45 degrees is assumed.

In this case, to maintain the rate of change in the drive force (F3/F1) between 0.75 and 1.25 inclusive, it is necessary to set L2/L1 between 0.2 and 0.9 inclusive.

In this case, to maintain the rate of change in the drive force (F3/F1) between 0.9 and 1.1 inclusive, it is necessary to set L2/L1 between 0.5 and 0.7 inclusive.

The case of a structure in which the end effector 7 is flexible at a flex angle φ up to 60 degrees is also assumed.

In this case, to maintain the rate of change in the drive force (F3/F1) between 0.75 and 1.25 inclusive, it is necessary to set L2/L1 between 0.4 and 0.8 inclusive.

The first embodiment described above leads to the following effect.

In the treatment tool 1 according to the first embodiment, in the state of sandwiching the shaft 83, the first and second wires 1226 and 1227 are arranged along the outer circumferential surfaces 831, respectively. In the state of being separate from each other in a distance smaller than the diameter of the specific circle that is formed by the outer circumferential surfaces 831, each of the first and second wires 1226 and 1227 extends toward the distal end of the end effector 7.

This makes it possible to sufficiently ensure a length dimension at the time when the first and second wires 1226 and 1227 are joined to the end effector 7. In other words, it is possible to increase strength of joining of the first and second wires 1226 and 1227 to the end effector 7. Thus, it is possible to, in the state where a large force is being applied to the first end second wires 1226 and 1227, preferably maintain joining of the first and second wires 1226 and 1227 to the end effector 7.

In the treatment tool 1 according to the first embodiment, the distance smaller than the diameter of the specific circle described above is set at a distance that maintains the state where the first and second wires 1226 and 1227 abut on the outer circumferential surfaces 831 even when the end effector 7 flexes toward, for example, the first flex direction Ar3 or the second flex direction Ar4 at a flex angle up to approximately 90 degrees with respect to the center axis Ax.

In other words, because the first and second wires 1226 and 1227 abut on the outer circumferential surfaces 831 even when the end effector 7 flexes at a flex angle up to approximately 90 degrees, it is possible to realize a structure that cause no sagging in the first and second wires 1226 and 1227. Thus, it is possible to, when the end effector 7 makes contact with living tissue, or the like, prevent the end effector 7 from flexing easily due to the force applied to the end effector 7 and provide treatment preferably.

In the treatment tool 1 according to the first embodiment, the first and second wires 1226 and 1227 are fixed to the end effector 7 using the holder 17 and the fall stopper 18. It is thus possible to join the first and second wires 1226 and 1227 to the end effector 7 using a simple structure.

In the treatment tool 1 according to the first embodiment, in the state where the specific plane on which the link mechanism 113 is movable is being orthogonal to the sixth rotation axis Rx6, the link mechanism 113 and the shaft 83 are superimposed along the sixth rotation axis Rx6. In other words, the link mechanism 113 and the shaft 83 are provided in approximately the same positions in a direction along the center axis Ax.

This makes it possible to shorten the length of the portion on the distal end side Ar1 with respect to the sixth rotation axis Rx6 and reduce the size of the distal end part of the treatment tool 1. Reducing the size of the distal end part of the treatment tool 1 makes it possible to provide fine treatment.

In the treatment tool 1 according to the first embodiment, the path 833 into which the four conductive cables CA1 are inserted is provided in the shaft 83. In other words, the four conductive cables CA1 are arranged in the portion at which the end effector 7 flexes via the shortest route passing through the sixth rotation axis Rx6.

Thus, to assemble the treatment tool, it is not necessary to perform complicated operations of adjusting the length of the conductive cables CA1, which makes it possible to increase easiness in assembling the treatment tool.

### Second Embodiment

A second embodiment will be described next.

In the following description, the same components as those of the above-described first embodiment are denoted with the same reference numbers and detailed description thereof will be omitted or simplified.

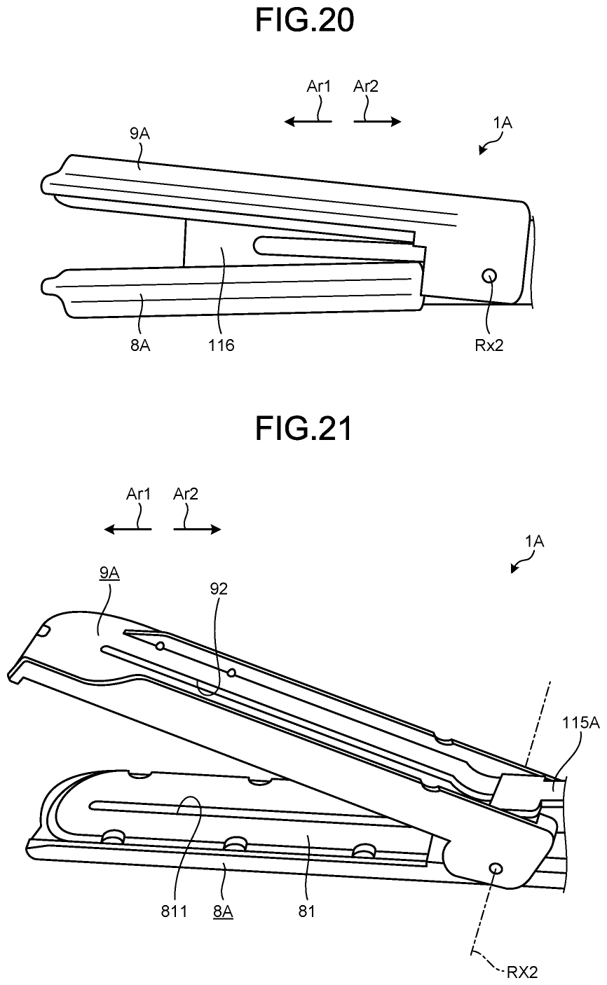

FIGS. 20 to 22 are diagrams illustrating a distal end part of a treatment tool 1A according to the second embodiment.

In the treatment tool 1A according to the second embodiment, a cutter 116 (FIG. 20) is added to the treatment tool 1 that is described in the above-described first embodiment.

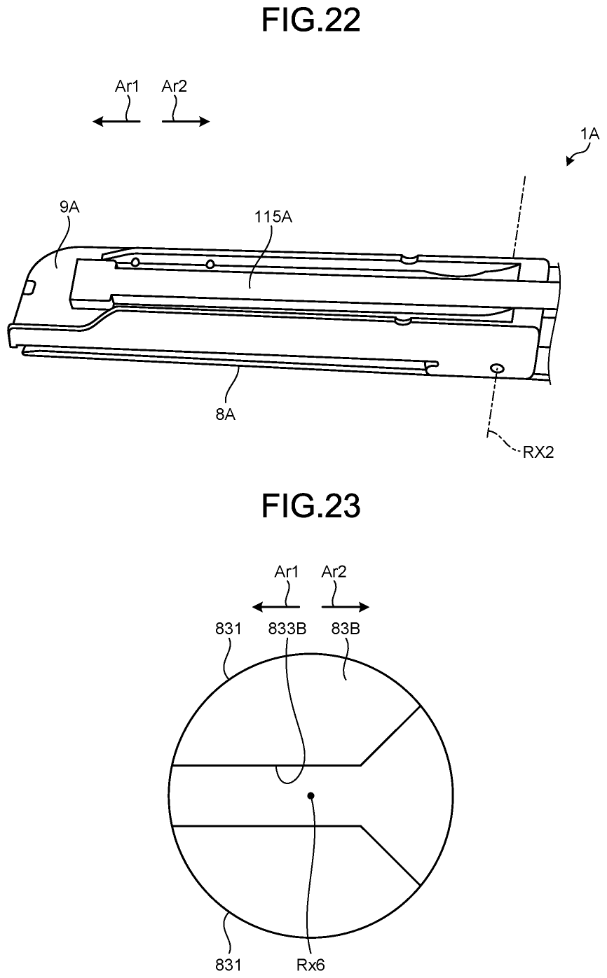

The distal end arm 115 according to the second embodiment will be referred to as a distal end arm 115A (FIG. 21 and FIG. 22) below for convenience of description. The first and second graspers 8 and 9 according to the second embodiment will be described as first and second graspers 8A and 9A.

The first grasper 8A is different from the first grasper 8 described in the above-described first embodiment in the following aspect.

As illustrated in FIG. 21, a groove 811 that extends linearly along the longitudinal direction of the first grasper 8A is provided in the first electrode 81.

The second grasper 9A is different from the second grasper 9 described in the above-described first embodiment in the following aspect.

A through-hole 92 that penetrates top and bottom surfaces of the second grasper 9A in FIG. 21 and that extends linearly along the longitudinal direction of the second grasper 9A is provided in the second grasper 9A.

The distal end arm 115A is different from the distal end arm 115 described in the above-described first embodiment in the following aspect.

In accordance with a forward or backward move of the open-close rod 112 along the center axis Ax, as illustrated in FIGS. 21 and 22, the distal end arm 115A slides on the top surface of the second grasper 9A in FIG. 21 toward the distal end side Ar1 or the proximal end side Ar2. The distal end arm 115A moves toward the distal end side Ar1 and accordingly the second grasper 9A rotates on the second rotation axis Rx2 in a direction in which the second grasper 9A gets close to the first grasper 8A (direction of closing). On the other hand, the distal end arm 115A moves toward the proximal end side Ar2 and accordingly the second grasper 9A rotates on the second rotation axis Rx2 in a direction in which the second grasper 9A separates from the first grasper 8A (direction of opening).

The cutter 116 is provided on an end portion of the distal end arm 115 on the distal end side Ar1 and extends into the groove 811 via the through-hole 92. In accordance with the move of the distal end arm 115A toward the distal end side Ar1, the cutter 116 moves toward the distal end side Ar1. The cutter 116 thus incises a subject region that is graspted between the first and second graspers 8A and 9A.

The second embodiment described above leads to an effect similar to that of the first embodiment described above.

### Third Embodiment

A third embodiment will be described next.

In the following description, the same components as those of the above-described first embodiment are denoted with the same reference numbers and detailed description thereof will be omitted or simplified.

FIG. 23 is a diagram illustrating a shaft 83B according to the third embodiment.

The shaft 83B according to the third embodiment is different from the shaft 83 described in the above-described first embodiment in the shape of the path 833.

The path 833 according to the third embodiment will be referred to as a path 833B (FIG. 23) below for convenience of description.

As illustrated in FIG. 23, a portion of the path 833B on the proximal end side Ar2 has a shape in which the width increases toward the proximal end side Ar2.

The third embodiment described above leads to the following effect in addition to an effect similar to that of the first embodiment described above.

The portion of the path 833B according to the third embodiment on the proximal end side Ar2 has the shape in which the width increases toward the proximal end side Ar2.

This makes it possible to reduce a pressure to be applied to the conductive cables CA1 from a corner of side walls of the path 833B on the proximal end side Ar2 when the end effector 7 flexes and inhibit deterioration of the conductive cables CA1 according to the flex.

### Fourth Embodiment

A fourth embodiment will be described next.

In the following description, the same components as those of the first embodiment and the third embodiment described above are denoted with the same reference numbers and detailed description thereof will be omitted or simplified.

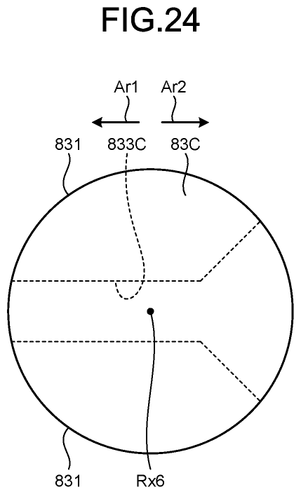

FIG. 24 is a diagram illustrating a shaft 83C according to the fourth embodiment.

The shaft 83C according to the fourth embodiment is different from the shaft 83B described in the above-described third embodiment in the shape of the path 833B.

The path 833B according to the fourth embodiment will be referred to as a path 833C (FIG. 24) below for convenience of description.

As described in FIG. 24, the path 833C is formed by not a groove like the path 833B described in the above-described third embodiment but a hole.

The fourth embodiment described above leads to an effect similar to those of the first embodiment and the third embodiment described above.

### Fifth Embodiment

A fifth embodiment will be described next.

In the following description, the same components as those of the first embodiment described above are denoted with the same reference numbers and detailed description thereof will be omitted or simplified.

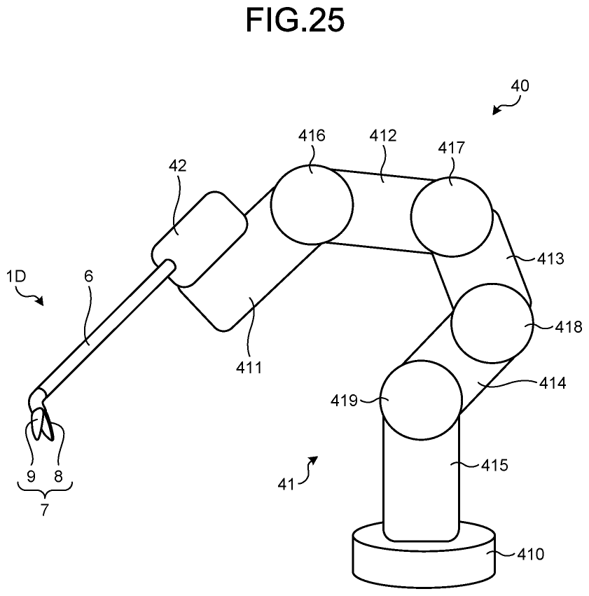

FIG. 25 is a diagram illustrating a medical device 40 according to a fifth embodiment.

As illustrated in FIG. 25, the medical device 40 according to the fifth embodiment has a configuration in which a treatment tool 1D that is configured differently from the treatment tool 1 descried in the above-described first embodiment is supported by a robot arm 41.

As illustrated in FIG. 25, the robot arm 41 includes a base 410, first to fifth arm parts 411 to 415, and first to fourth joints 416 to 419.

The base 410 is set on a floor, or the like, and supports the whole medical device 40.

The first to fifth arm parts 411 to 415 are connected in series with the first to fourth joints 416 to 419. The fifth arm unit 415 that is positioned at a proximal end among the first to fifth arm parts 411 to 415 is fixed onto the base 410. The treatment tool 1D is detachably connected to the first arm part 411 that is positioned at a distal end among the first to fifth arm parts 411 to 415.

The first to fourth joints 416 to 419 allow a pair of arm parts that are connected mutually among the first to fifth arm parts 411 to 415 to relatively rotate on axes that are different from one another. In other words, in the fourth embodiment, the treatment tool 1D is movable at four degrees of freedom. Note that the degrees of freedom are not limited to four degrees of freedom and the robot arm 41 may have a different number of degrees of freedom. In other words, the number of the first to fifth arm parts 411 to 415 and the number of the first to fourth joints 416 to 419 are not limited to the above-described numbers and may be other numbers.

Although not specifically illustrated in the drawings, actuators for causing a pair of arm parts that are connected mutually among the first to fifth arm parts 411 to 415 to rotate relatively are provided in the first to fourth joints 416 to 419, respectively. Each of the actuators is driven under the control of the external control device (not illustrated in the drawings).

As illustrated in FIG. 25, the treatment tool 1D includes an attachment-detachment unit 42 in addition to the sheath 6, the end effector 7, the first and second flex rods 1224 and 1225, the first and second wires 1226 and 1227, the open-close rod 112, and the link mechanism 113 that are described in the above-described first embodiment. Note that the first and second flex rods 1224 and 1225, the first and second wires 1226 and 1227, the open-close rod 112, and the link mechanism 113 are not shown in FIG. 25.

The attachment-detachment unit 42 is a part that is provided at a proximal end of the sheath 6 and that allows the treatment tool 1D to be attached to or to be detached from the robot arm 41 (the first arm part 411). Although not specifically illustrated in the drawings, an actuator that applies a drive force to the first and second flex rods 1224 and 1225 or the open-close rod 112 is provided in the attachment-detachment unit 42. The actuator is driven under the control of the external control device (not illustrated in the drawings). Accordingly, opening or closing of the second grasper 9 with respect to the first grasper 8 and the flex move of the end effector 7 with respect to the sheath 6 are performed.

The fifth embodiment described above leads to an effect similar to that of the first embodiment.

### Other Embodiments

Modes for carrying out the disclosure have been described; however, the disclosure should not be limited only by the first to fifth embodiments described above.

In the first to fifth embodiments described above, a high-frequency energy and a thermal energy are exemplified as the treatment energy that is applied to the subject region; however, the treatment energy is not limited thereto. It is possible to employ at least any one of a high-frequency energy, a thermal energy, and an ultrasound energy as the treatment energy. “Applying an ultrasound energy to the subject region” means applying ultrasound vibrations to the subject region.

The treatment tool according to the disclosure is not limited to the configuration of treat the subject region by applying the treatment energy to the subject region and covers forceps that only grasp a subject region.

According to the first to fifth embodiments, only one of the shafts 83, 83B and 83C is provided and the end effector 7 is flexible on only the sixth rotation axis Rx6; however, the flex is not limited thereto. The end effector 7 may be configured such that the end effector 7 is flexible on each of a plurality of rotation axes.

According to the first to fifth embodiments described above, the link mechanism 113 includes the two arms that are the proximal end arm 114 and the distal end arm 115; however, the link mechanism 113 is not limited thereto. The link mechanism may include at least three arms.

Note that the following configuration belongs to the scope of the disclosure.

(1) A treatment tool including an insertion tube that is tubular and that is at least partly inserted into a body; a distal end part that is provided at a distal end of the insertion tube and that is flexible with respect to the insertion tube; a shaft that is arranged in the distal end part and, when viewed from a direction along a rotation axis on which the distal end part is caused to flex with respect to the insertion tube, has an outer circumferential surface that is positioned on a circumference of a specific circle on the rotation axis; and a pair of transmitters each of which is inserted into the insertion tube, is fixed to the distal end part, and transmits a drive force that causes the distal end part to flex with respect to the insertion tube, wherein the transmitter includes a wire and a rod that is connected to a proximal end of the wire and that has a rigidity higher than that of the wire.

(2) The treatment tool according to (1), wherein the transmitter further includes a connector that connects the wire and the rod.

The case where the first and second rods 1224 and 1225 are omitted and each of the first and second wires 1226 and 1227 is extended to the proximal end side Ar2 and is fixed directly to the first and second drivers 1222 and 1223 is assumed.

In this case, because the length dimensions of the first and second wires 1226 and 1227 increase, amounts of stretch of the first and second wires 1226 and 1227 caused by a large force applied to the first and second wires 1226 and 1227 increase. As a result, sagging tends to occur in the first and second wires 1226 and 1227 and, when the end effector 7 makes contact with living tissue, or the like, the end effector 7 easily flexes because of the force applied to the end effector 7.

On the other hand, according to (1) and (2) described above, using the first and second flex rods 1224 and 1225 makes it possible to reduce the length dimensions of the first and second wires 1226 and 1227. In other words, it is possible to reduce the amount of stretch of the first and second wires 1226 and 1227 caused by the large force applied to the first and second wires 1226 and 1227. As a result, sagging does not tend to occur in the first and second wires 1226 and 1227 and it is possible to prevent the end effector 7 from easily flexing when the end effector 7 makes contact with living tissue because of the force applied to the end effector 7.

(3) A treatment tool including an insertion tube that is tubular and that is at least partly inserted into a body; a pair of jaws that are provided at a distal end of the insertion tube, that are flexible with respect to the insertion tube, and that grasp living tissue by opening and closing with each other; a shaft that, when viewed from a direction along a first pivot axis on which the jaws are caused to flex with respect to the insertion tube, has an outer circumferential surface that is positioned on a circumference of a specific circle on the first pivot axis; a rod that is inserted into the insertion tube and that moves forward and backward along a longitudinal axis of the insertion tube; and a link mechanism that is linked to a distal end of the rod and that includes a plurality of arms that are linked with each other movably within a specific plane, wherein the link mechanism is linked to the rod flexibly on a second pivot axis and is linked to one of the jaws flexibly on a third pivot axis and a rate of a length L2 from the first pivot axis to the third pivot axis to a length L1 from the second pivot axis to the third pivot axis is set to enable a rate of a second drive force that is transmitted from the link mechanism to the one of the jaws to a first drive force that is transmitted to the rod to be a rate within a specific range.

(4) The treatment tool according to (3), wherein the rate within the specific range is between 0.75 and 1.25 inclusive and the rate of the length L2 to the length L1 is set between 0.2 and 0.9 inclusive.

(5) The treatment tool according to (3), wherein the rate within the specific range is between 0.9 and 1.1 inclusive and the rate of the length L2 to the length L1 is set between 0.5 and 0.7 inclusive.

(6) The treatment tool according to (3), wherein the rate within the specific range is between 0.75 and 1.25 inclusive and the rate of the length L2 to the length L1 is set between 0.4 and 0.8 inclusive.

According to (3) to (6) described above, it is possible to easily keep the rate of the second drive force to the first drive force at a rate within the specific range and easily set a grasp force by which a target region is grasped at an intended grasp force.

According to the treatment tool according to the disclosure, it is possible to reduce the size.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
an insertion tube that is tubular, the insertion tube being configured to be at least partly inserted into a body;
a pair of jaws that are provided at a distal end of the insertion tube and that are flexible with respect to the insertion tube, the pair of jaws being configured to grasp living tissue by opening and closing each other;
a pin that is provided on a rotation axis on which the jaws are caused to rotate with respect to the insertion tube;
a shaft that has an outer circumferential surface and in which a hole into which the pin is inserted, the outer circumferential surface being positioned on a circumference of a specific circle on the rotation axis when viewed from a direction along the rotation axis on which the jaws are caused to flex with respect to the insertion tube;
a rod configured to be inserted into the insertion tube and move forward and backward along a longitudinal axis of the insertion tube; and
a link mechanism that is linked to a distal end of the rod and that includes a plurality of arms linked with each other movably within a specific plane,
the link mechanism and the shaft being superimposed along the rotation axis with the specific surface being intersecting with the rotation axis.

2. The treatment tool according to claim 1, wherein the shaft is provided in one of the jaws.

3. The treatment tool according to claim 1, wherein the jaws is configured to open and close each other along the rotation axis.

4. The treatment tool according to claim 1, wherein the jaws is configured to open and close each other when a drive force is transmitted to the jaws via the link mechanism according to a forward move and a backward move of the rod along the longitudinal axis of the insertion tube.

5. The treatment tool according to claim 4, wherein the arms include
a proximal end arm that is linked to the rod; and
a distal end arm to which the proximal end arm and one of the jaws are linked.

6. The treatment tool according to claim 5, wherein, when viewed from a direction along the rotation axis, the shaft is positioned between the proximal end arm and the distal end arm.

7. The treatment tool according to claim 1, wherein a cutter is provided at a distal end of the link mechanism, and
the cutter is configured to incise the living tissue that is grasped between the jaws when a drive force is transmitted to the jaws via the link mechanism according to forward and backward move of the rod along the longitudinal axis of the insertion tube.

8. The treatment tool according to claim 1, wherein the jaws is configured to apply a treatment energy to the living tissue to treat the living tissue.

9. The treatment tool according to claim 8, wherein an electrode configured to apply a high-frequency energy to the living tissue according to a supplied power is provided in each of the jaws, the high-frequency energy being the treatment energy.

10. The treatment tool according to claim 8, wherein a heater configured to generate heat according to a supplied power to apply a thermal energy to the living tissue is provided in at least one of the jaws, the thermal energy being the treatment energy.

11. The treatment tool according to claim 1, wherein a connector is provided in the insertion tube and the pin is mounted via the connector.

12. The treatment tool according to claim 1, wherein the link mechanism is movable within a plane orthogonal to the rotation axis.
